(19) United States
(12) Patent Application Publication
(10) Pub. No.: US 2010/0196476 A1
(43) Pub. Date: Aug. 5, 2010

(54) PROCESS FOR THE PREPARATION AND PHARMACEUTICAL FORMULATIONS FOR 4-QUINOLINONES AND QUINOLINES AND USE THEREOF

(75) Inventors: Arlene Goncalves Correa, Sao Carlos (BR); Patricia Tambarussi Baraldi, Itapolis (BR); Andreimar Martins Soares, Ribeira Preto (BR)

Correspondence Address: ARENT FOX LLP, 1050 CONNECTICUT AVENUE, N.W., SUITE 400, WASHINGTON, DC, 20036, US

(73) Assignee: FUNDACAO UNIVERSIDADE FEDERAL DE SAO CARLOS, Sao Carlos-SP (BR)

(21) Appl. No.: 12/602,347

(22) PCT Filed: May 23, 2008
(86) PCT No.: PCT/BR08/00151

§ 371 Date: Apr. 16, 2010

(30) Foreign Application Priority Data
May 28, 2007 (BR) P10701664-6

(51) Int. Cl. A61K 31/47; A61K 31/496; C07D 215/48; C07D 215/233; C07D 401/10; A61K 9/28; A61P 29/00; A61P 37/08; A61P 37/06; A61P 43/00; A61P 7/04; A61P 7/02; A61P 7/00; A61P 11/10

(52) U.S. Cl. 424/474; 514/253.08; 546/156; 546/153; 544/363; 514/312

(57) ABSTRACT

The present invention describes novel 4-quinolinones corresponding to formula (I) and quinoline derivatives thereof as formula (II), process of preparation thereof, pharmaceutical formulations comprising said 4-quinolinones and pharmaceutical application thereof for diseases related to disorders of white blood cells, such as inflammatory and autoimmune diseases, including rheumatism, as well as the use as anti-coagulant, antivenin, analgesic and for antithrombotic purposes.

### FIELD OF THE INVENTION

The present invention refers to the field of 4-quinolinones and derived quinolines, to be used in pharmaceutical formulations as synthetic protease inhibitors, such as haemorrhagic metalloprotease resulting from bites of the snakes of the Bothrops genus and other applications.

### BACKGROUND OF THE INVENTION

Snake venoms comprise complex protein mixtures including phospholipases A₂, myotoxins, haemorrhagic metalloprotease, coagulant serine protease, cytotoxins, cardiotoxins and others. The pathophysiology of poisoning by snakes involves a complex series of events that depend on the combined action of these components. (Gutiérrez, J. M. “Comprendiendo los venenos de serpientes: 50 años de investigaciones en América Latina” [Understanding snake venoms: 50 years of research in Latin América]. Rev. Biol. Trop., 50:377, 2002). Both phospholipases A₂ and proteases are abundantly present in snake venom, as well as having a digestive role in the hydrolysis of phospholipids and proteins. These enzymes may present a broad variety of pharmacological activities, such as neurotoxicity, myotoxicity as well as oedematogenic, haemorrhagic and coagulant activities, amongst others (Gutiérrez, J. M. & Lomonte, B. “Phospholipase A₂, myotoxins from Bothrops snake venoms”. Toxicon, 33:1405, 1995.; Ownby C. L. J. “Structure, function and biophysical aspects of the myotoxins from snake venoms”. Toxicol.-Toxins Review, 17:213, 1998.; Ownby, C. L.; Araujo, H. S. S.; White, S. P. & Fletcher, J. E. “Lysine 49 phospholipase A₂ proteins”. Toxicon, 37: 411, 1999.; Soares, A. M.; Fontes, M. R. M. & Giglio, J. R. Phospholipase A₂ myotoxins from Bothrops snake venoms: Structure-Function relationship. Review. Curr. Org. Chem., 8: 1677, 2004.).

Pharmacological studies have demonstrated that the extracts and fractions of certain plants used in traditional medicine possess anti-inflammatory, antiviral and antivenin properties (Martz, W. “Plants with a reputation against snakebite”. Toxicon, 30:1131, 1992.; Mors, W. B.; Nascimento, M. C.; Pereira, B. M. R. & Pereira, N. A. “Plant natural products active against snake bite—the molecular approach”. Phytochemistry, 55: 627, 2000.; Soares, A. M.; Ticli, F. K.; Marcussi, S.; Lourenço, M. V.; Januário, A. H.; Sampaio, S. V.; Giglio, J. R.; Lomonte, B. & Pereira, P. S. “Medicinal plants with inhibitory properties against snake venoms”. Curr. Med. Chem., 12:2625, 2005.; Soares, A. M.; Januário, A. H.; Lourenço, M. V.; Pereira, A. M. S.; Pereira, P. S. Drugs Future 29:1105, 2004.)

Patent application US2004/0242639 presents the activity of a phospholipase C inhibitor as therapy for inflammatory diseases. On the other hand, quinolinones with N-heteroamino in position 5 of the quinolinone ring described in publication WO94/10163 (and the corresponding U.S. Pat. No. 5,646,163 and Brazilian patent applications BR 9307347 and BR 9507553) present antimicrobial activity, while the quinolinones substituted in position 3 described in this invention present inhibitor activity for phospholipase C.

Patent document EP0304158 claims quinolones possessing anti-bacterial activity with variations of the substitutes in positions and 3, while patent application JP2000273086 describes quinolinones only substituted in position 2.

Despite the structures described in publication CN1817880A being of the type 4-quinolinones, the substitute in position 2 differs from those included in the claims herein.

Patent document WO01/53266 describes type 4-quinolinone structures substituted in position 3 that are useful for treating diseases associated to white blood cell disorders such as autoimmune and inflammatory diseases but, however, are distinct from the structures proposed in the present invention.

The US patent application published as US2003/0124120 describes quinolinones with antagonistic activity in vitronectin receptors and presents variants that may be either in positions 2 or 3.

Certain 2-quinolinone compounds having serine protease activity were described in U.S. Pat. No. 6,855,726. Yet other quinolinone structures were claimed in patent document WO01/70698.

The international publication WO2004/007461 describes a method for the treatment or prophylaxis of a neurological condition—more specifically neurodegenerative disorders—that consists administering an effective quantity of a composition of the formula (I) to a patient requiring treatment. The standard skeleton of the molecule that is the object of that publication is similar to the compounds of the present invention but, however, despite the many variations of the substitutes described, no skeleton suggests or describes compounds similar to the compounds described herein.

In the same manner, U.S. Pat. No. 5,444,071 and patent application US2006/0217322 describe compounds having pharmaceutical activity with basic skeletons similar to those of the compounds proposed herein but none of these structures anticipates those of the present invention.

U.S. Pat. No. 5,102,892 describes compounds with a quinoline structure similar to those described in the present invention but, however, varying the position 4—from Oxygen to Nitrogen.

International publication WO2006/068617 presents a methodology for the preparation of enamine using dimethyl acetylenedicarboxylate (DMAD) but, however, the input materials and products obtained are different from those that constitute the object of the present invention. No intermediate similar to the compounds of that invention is suggested or described.

The publications BR1100774, WO02/200625 and WO97/21680 describe other classes of quinolines different from those comprising the object of the present invention.

In the compounds described in U.S. Pat. No. 5,789,419, U.S. Pat. No. 4,412,075 and U.S. Pat. No. 4,593,101, the skeletons present a difference compared to the double conjugate that is not found in the compounds of the present invention and neither use the conjugate system proposed herein. Furthermore, patent document U.S. Pat. No. 5,789,419 describes compounds intended to treat bronchial e circulatory system disorders.

The structures described in patent EP1458718 are of the type 2-quinolinones and the activity is present in various receptors.

U.S. Pat. No. 4,859,669, U.S. Pat. No. 6,271,416, CN1594295, U.S. Pat. No. 6,855,726, patent application US2005/0054672, publications EP1574501, EP1097139B1, EP1270006 and patent application US 2005/0209247 describe 2-quinolinones structurally distinct from those object of the present invention.

Patent documents EP1245566 and U.S. Pat. No. 6,645,983 describe 4-quinolinones but, however, the structure described does not include a substitute in position 2 and the biological activities are respectively described as being anti-microbial and intended for the treatment of cancers.

Publication EP0251308 describes a fluoroquinolinone as substitute in positions 2 and 3 of the quinolinone system. R₂ is H or S linked to the N of the ring by an ethylene bridge.

Thus, the literature shows that despite considerable technological development, there is still great need for new 4-quinolinones obtained through processes using derivates of borane as ester reducing agents in the presence of enone, with these compounds being useful as protease, lipase and phospholipase inhibitors as well as having other medicinal applications such as for disorders affecting the haemostatic system or white blood cells. The process for the preparation, the compounds and the pharmaceutical formulations of the 4-quinolinones and their quinoline derivates are described and claimed herein.

### SUMMARY OF THE INVENTION

Overall, the present invention refers to derivates of 4-quinolinone in accordance with the formula below:

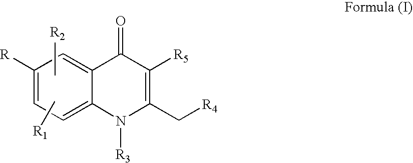

Whereby:

### R═H or OCH₃

R₁ and R₂ are selected independently of each other, with H, OH, an alkyl group of C₁-C₄, an alkoxy group of C₁-C₅, a —OCO—R₇ group, and a group derived from a saccharide, optionally R₁ and R₂ together forming a methylenedioxy group, a phenyl group or a phenyl group substituted in 1 and 3 with groups selected from H, an alkoxy group in C₁-C₄, a —OCOR₇ group, a —O—SO₂—R₇ group, halogen, an alkyl or CF₃ group, and —NR₇R₈ group, in which R₇ and R₅ are selected independently of each other, from hydrogen, alkyl group in C₁-C₅, alkenyl group, alkyl phenyl group (C₁-C₄), dimethylamine, rings with 4-6 member heterocycles, optionally with one or more heteroatoms selected from oxygen, nitrogen and sulphur or a methylpiperazinyl group;
R₃ is selected from H, alkyl group in C₁-C₄, alkenyl group, a —CO—R₈ group and a -A-R₉ group, —CO₂R_{9′} group in which R₉′ is a benzyl group, branched or linear alkyl group, p-methoxy benzyl group, —NH₂, —CHCH₂CH₂, R₈ is an alkyl group in C₁-C₄; A is an alkylene group in C₁-C₄; R₉ is selected from heterocycle groups with 5 or 6 members containing 1 to 4 heteroatoms of oxygen, sulphur and nitrogen, CN, hydroxyl, —COOR₁₀ and CONR₁₁R₁₂ groups, a —NR₁₃R₁₄ group, a —COR₁₅ group and a OSO₂R₁₆ group;
R₁₀, R₁₁, R₁₂, R₁₄ and R₁₅ are independently selected from hydrogen, alkyl groups in C₁-C₄, halogen and alkyl phenyl group (C₁-C₄), R₁₆ is selected from the phenyl group and the alkyl phenyl group (C₁-C₄);
R₄═OH, halogens, alkoxy group in C₁-C₆, alkoxy benzyl group, —CO—R₁₇ in which R₁₇ is alkyl C₁-C₆ or p-methoxy benzyl, —O—SO₂—R₇′ in which R₇′ is an alkyl group or CF₃ group, group derived from saccharide; R₅ is H, halogen, phenyl group or phenyl substituted 1 or 3 times with groups selected from H, alkoxy groups C₁-C₄, a —OCOR₇ group, a —O—SO₂—R₇′ group in which R₇′ is an alkyl group or CF₃ group, benzylamine group, and group derived from saccharide, alkyl group, —COOH, or salts, hydrates and pharmacologically acceptable pro-pharmacons.

The compounds of Formula (I) produce the quinolines of Formula (II) in the presence of a K₂CO₃ base and alkylating agents:

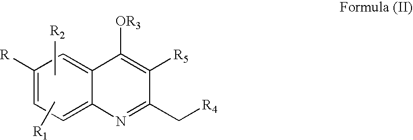

Whereby:
R═H or OCH₃
R₁ and R₂ are selected independently of each other, with H, OH, a group R₄═OH, halogens, an alkoxy group in C₁-C₆, alkoxy benzyl group, —CO—R₁₇ in which R₁₇ is alkyl C₁-C₆ or p-methoxy benzyl, —O—SO₂—R₇′ in which R₇′ is an alkyl group or CF₃ group, group derived from a saccharide; R₅ is H, halogen, phenyl group or phenyl substituted 1 or 3 times with groups selected from H, alcoxy C₁-C₄ groups, a —OCOR₇ group, a —O—SO₂—R₇′ group in which R₇′ is an alkyl group or CF₃ group, benzylamine group, and group derived from a saccharide, alkyl group, —COOH, or salts, hydrates and pharmacologically acceptable pro-pharmacons in C₁-C₄, an alcoxy group in C₁-C₅, a —OCO—R₇ group, and a group derived from a saccharide, optionally R₁ and R₂ together forming a methylenodioxy group, a phenyl group or phenyl group substituted in 1 to 3 with groups selected from H, an alkoxy C₁-C₄ group, a —OCOR₇ group, a —O—SO₂—R₇ group, halogen, alkyl group or CF₃ group and —NR₇R₈ group, in which R₇ and R₈ are selected independently of each other, from hydrogen, alkyl group in C₁-C₅, alkenyl group, alkyl phenyl group (C₁-C₄), dimethylamine, 4-6 heterocycle member rings optionally with one or more heteroatoms selected from among oxygen, nitrogen and sulphur or a methylpiperazinyl group;
   R₃ is selected from H, alkyl group in C₁-C₄, alkenyl group, a —CO—R₈ group and a -A-R₉ group, —CO₂R₈′ group in which R₉′ is a benzyl group, branched or linear alkyl group, p-methoxy benzyl group, —NH₂, —CHCH₂CH₂; R₈ is an alkyl group in C₁-C₄; A is an alkylene group in C₁-C₄; R₉ is selected from heterocycle groups with 5 or 6 members containing 1 to 4 heteroatoms of oxygen, sulphur, nitrogen, CN, hydroxyl, —COOR₁₀ and CONR₁₁R₁₂ groups, a —NR₁₃R₁₄ group, a —COR₁₅ group and a OSO₂R₁₆ group; R₁₀, R₁₁, R₁₂, R₁₄ and R₁₅ are independently selected from hydrogen, alkyl groups in C₁-C₄, halogen and alkyl phenyl group (C₁-C₄), R₁₆ is selected from the phenyl group and the alkyl phenyl group (C₁-C₄);
   R₄═OH, halogens, alkoxy group in C₁-C₆, alkoxy benzyl group, —CO—R₁₇ in which R₁₇ is alkyl C₁-C₆ or p-methoxy benzyl, —O—SO₂—R₇′ in which R₇′ is an alkyl group or CF₃ group, group derived from saccharide; R₅ is H, halogen, phenyl group or phenyl substituted 1 or 3 times with groups selected from H, alkoxy groups C₁-C₄, a —OCOR₇ group, a —O—SO₂—R₇′ group in which R₇′ is an alkyl group or CF₃ group, benzylamine group, and group derived from saccharide, alkyl group, —COOH, or salts, hydrates and pharmacologically acceptable pro-pharmacons.

The invention also refers to a process for preparing the compounds of formula (I) and formula (II), with the above mentioned processes comprising the stages of:
a) make anilin (compound 3) or 4-methoxyanilin (compound 4) react with dimethyl acetylenedicarboxylate (DMAD) in methanol at 55° C., obtaining 2-(phenylamine)-dimethyl maleate or 2-anilin-2-dimethyl butenodioate (compound 5) or 2-(4-methoxyphenylamine)-dimethyl maleate or 2-(4-methoxyanilin)-2-dimethyl butenodioate (compound 6).

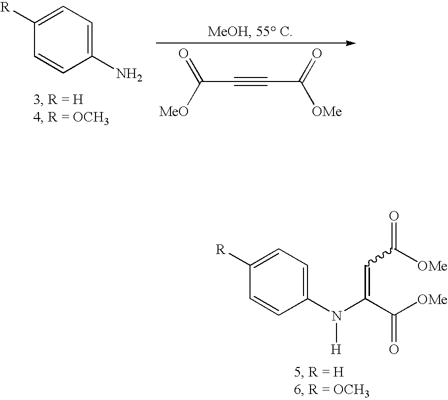

b) make compounds 5 or 6 react by reflux with diphenylether (DFE) to produce 4-oxo-1,4-dihydro-2-quinoline methyl carboxylate (compound 7) or 6-methoxy-4-oxo-1,4-dihydro-2-quinoline methyl carboxylate (compound 8);

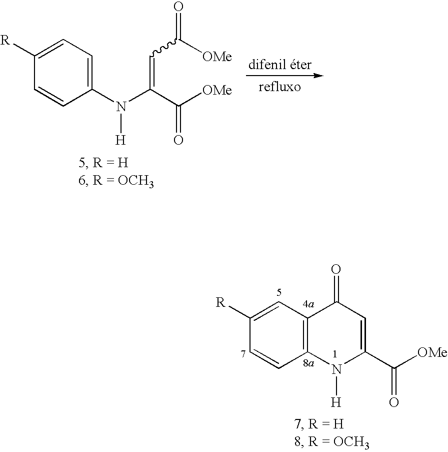

c) make compounds 7 and 8 react with the borane-dimethyl sulphide (BH₃.5Me₂) complex, obtaining compounds 1,2-hydroxymethyl-4-quinolinone and 2,2-hydroxymethyl-6-methoxy-4-quinolinone.

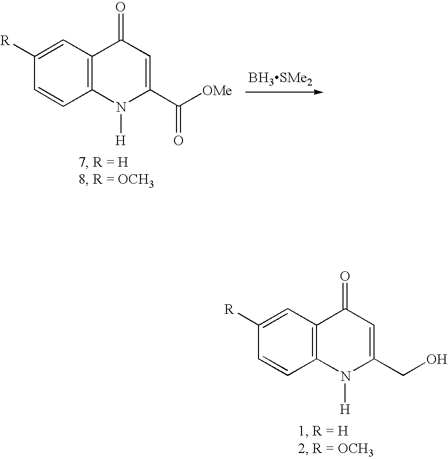

d) make compound 2 react with: 1) K₂CO₃ (potassium carbonate), DMF (dimethyl formamide), EtBr (Ethidium bromide) at 80% obtaining 4-ethoxy-2-hydroxymethyl-6-methoxyquinoline (compound 10) 2) NaH (sodium hydrate), EtBr 80%, or 4-ethoxy-2-ethoxymethyl-6-methoxyquinoline (compound 11).

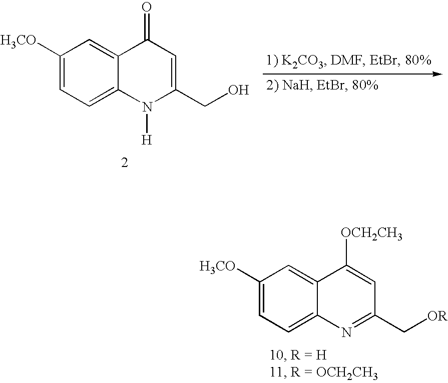

The invention provides compounds 1, 2, 10, 11 and the general formula (I) and (II) as being protease, lipase, phospholipase and enzyme inhibitors.

The invention provides compounds 1, 2, 10, 11 and the general formula (I) and (II) to be used in the broad aspect of inflammatory, antirheumatic, analgesic, autoimmune, antivenin, antithrombotic, anti-allergic and expectorant activities, as well as white blood cell disorders and haemostatic system disorders amongst other possible pharmaceutical applications.

The invention provides compositions, formulations or medicines containing effective amounts of compounds 1, 2, 10, 11 and the general formula (I) and (II) or their pharmaceutically acceptable salts.

The invention also provides compositions including the compounds 1, 2, 10, 11 and the general formula (I) and (II) to be used in the broad aspect of inflammatory, antirheumatic, analgesic, autoimmune, antivenin, antithrombotic, anti-allergic and expectorant activities, as well as white blood cell disorders and haemostatic system disorders amongst other possible pharmaceutical applications.

The invention also provides pharmaceutical formulations prepared in the form of pills, coated pills, capsules, inhalable powder, effervescent tablets, sublingual pills, syrups and oral solutions, injectable solutions, ointments, creams, gels and other pharmaceutical preparations known in pharmaceutical techniques.

The invention also provides the administration of the above mentioned formulations by oral, rectal, topical or parenteral route, with the active principle in a quantity not less than 0.001% of the composition's final weight together with at least one pharmaceutically appropriate excipient.

The invention also provides pharmaceutical formulations that comprise: a) a compound of general formula (I) as an active principle in a quantity not less than 0.001% of the composition's final weight, and b) at least one pharmaceutically appropriate excipient.

The invention also provides pharmaceutical formulations that comprise: a) a compound of general formula (II) as an active principle in a quantity not less than 0.001% of the composition's final weight, and b) at least one pharmaceutically appropriate excipient.

Furthermore, the invention provides pharmaceutical formulations that comprise the 4-quinolinones and quinoline derivates of the present invention to be administered to animals and/or humans.

### BRIEF DESCRIPTION OF THE DRAWINGS

FIG. 1 in annex illustrates the 4-quinolinone derivates tested for antivenin activity. The biological assays were also used to assess two commercial quinolinones, 12 (4-methoxy-2-quinolinecarboxylic acid) and the ciprofloxacine (1-ciclopropyl-6-fluoro-1,4-dihydro-4-oxo-7-(1-piperazinyl)-3-quinolinecarboxylic acid) (13), a fluoroquinolinone with a much described antibiotic activity.

FIG. 2 in annex is a bar graph for the compounds of FIG. 1, prepared for snake venom. FIG. 2A shows the results for 1. B. jararacussu; 2. B. moojeni. FIG. 2B shows the results for 3. B. alternatus; 4. B. jaracussu BjussuMP-1, whereby m/m=mass/mass.

FIG. 3 in annex shows the haemorrhagic, photolytic and coagulant inhibitory activity of compound 2. FIG. 3A: Effect of compound 2 on the haemorrhage induced by the venom of Bothrops and the isolated metalloprotease. FIG. 3B: Effect of compound 2 on the proteolytic activity induced by the venom of Bothrops and the isolated metalloprotease. FIG. 3C: Effect of the coagulant activity of compound 2 induced by the venoms of Bothrops and Crotalus and the isolated serine protease. The results are expressed by the mean±standard deviation (SD) (n=6).

FIG. 4A in annex shows the fibrinogenolytic inhibitory activity induced by metalloprotease and serine protease enzymes. FIG. 4B shows by means of SDS-PAGE that there is no evidence of the proteolytic degradation of venom proteins.

FIG. 5 in annex shows by graph the myotoxicity, oedema and phospholipase inhibitory activity of compound 2. FIG. 5A: Effect of compound 2 on the myotoxicity induced by the venom of B. jararacussu and isolated myotoxins (BthTX-I and II). FIG. 5B: Effects of compound 2 on the oedema inducing activity of the venom of B. jararacussu and isolated PLA-₂s (basic Lys49 BthTX-I, basic Asp49 BthTX-II and Asp49 BthA-1-PLA₂ acid). FIG. 5C: Effect of the phospholipase activity induced by the venom of B. jararacussu and C. d. terrificus and isolated PLA₂s (BthTX-II and CB). The results are expressed by the mean±standard deviation (SD) (n 6).

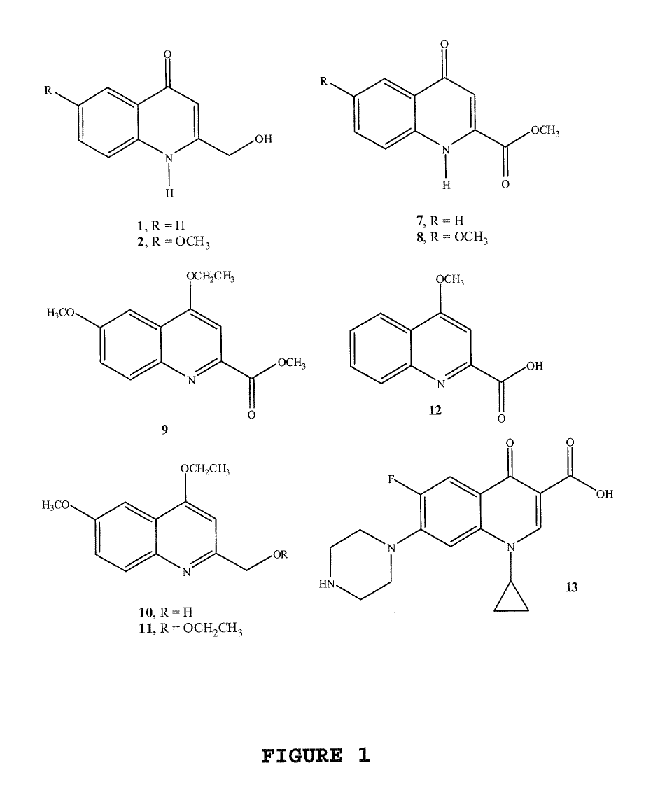

### DETAILED DESCRIPTION OF THE INVENTION

A first aspect of the invention are the 4-quinolinone compounds and quinoline derivates in accordance to formula (I) and formula (II).

A second aspect of the present invention relates to the preparation process of the 4-quinolinones with pharmacological activity and, more specifically, activity against the venom of the Bothrops genus snakes.

A third aspect of the invention are the medicinal formulations containing an efficient quantity of a 4-quinolinone compound (or a quinoline derivate) of the invention.

A fourth aspect of the invention is the use of the formulated compounds as inhibitors of metalloprotease, serine protease, autoimmune and inflammatory diseases including rheumatism as well as for anti-coagulant, antivenin, analgesic and antithrombotic purposes amongst other pharmaceutical applications.

According to the process of the invention, the 4-quinolinones of the invention, 2-hydroxymethyl-1,4-dihydro-4-quinolinone (compound 1) and 2-hydroxymethyl-6-methoxy-1,4-dihydro-4-quinolinone (compound 2), are synthetised using a methodology that uses BH₃.5Me₂ as reducer agent and uses 4-oxo-1,4-dihydro-2-quinoline methyl carboxylate (compound 3) and 6-methoxy-4-oxo-1,4-dihydro-2-quinoline methyl carboxylate (compound 4) as initial material. Furthermore, 1 and 2 respectively provide the quinoline compounds 10 and 11 by reaction with K₂CO₃, DMF, EtBr 80% followed by NaH, EtBr 80%.

The reduction reaction using borane-dimethyl sulphide occurs selectively in the ester group rather than in the enone group of the 4-quinolinone system. Apart from these novel compounds, it is further possible to prepare intermediates and derivates.

The biological evaluation of these compounds was for antivenin activity. Snake venoms comprise complex mixtures of proteins including phospholipases A₂ (PPA₂), myotoxins, haemorrhagic metalloproteases, coagulant serine proteases, cytotoxins, cardiotoxins and others. The pathophysiology of snake poisoning involves a complex series of events that depends on the combined action of these components (Gutiérrez, J. M. “Comprendiendo los venenos de serpientes: 50 años de investigaciones en América Latina” [Understanding snake venoms: 50 years of research in Latin América]. Rev. Biol. Trop., 50:377, 2002). Both phospholipases A₂ and proteases are abundantly present in snake venom, as well as having a digestive role in the hydrolysis of phospholipids and proteins. These enzymes may present a broad variety of pharmacological activities, such as neurotoxicity, myotoxicity as well as oedematogenic, haemorrhagic and coagulant activities, amongst others (Gutiérrez, J. M. & Lomonte, B. “Phospholipase A₂, myotoxins from Bothrops snake venoms”. Toxicon, 33:1405, 1995.; Ownby C. L. J. “Structure, function and biophysical aspects of the myotoxins from snake venoms”. Toxicol.-Toxins Review, 17:213, 1998.; Ownby, C. L.; Araujo, H. S. S.; White, S. P. & Fletcher, J. E. “Lysine 49 phospholipase A₂ proteins”. Toxicon, 37: 411, 1999.; Soares, A. M.; Januário, A. H.; Lourenço, M. V.; Pereira, A. M. S.; Pereira, P. S. Drugs Future 29:1105, 2004).

The preparation processes of the compounds of the present invention are summarily described in Diagram 1 below:

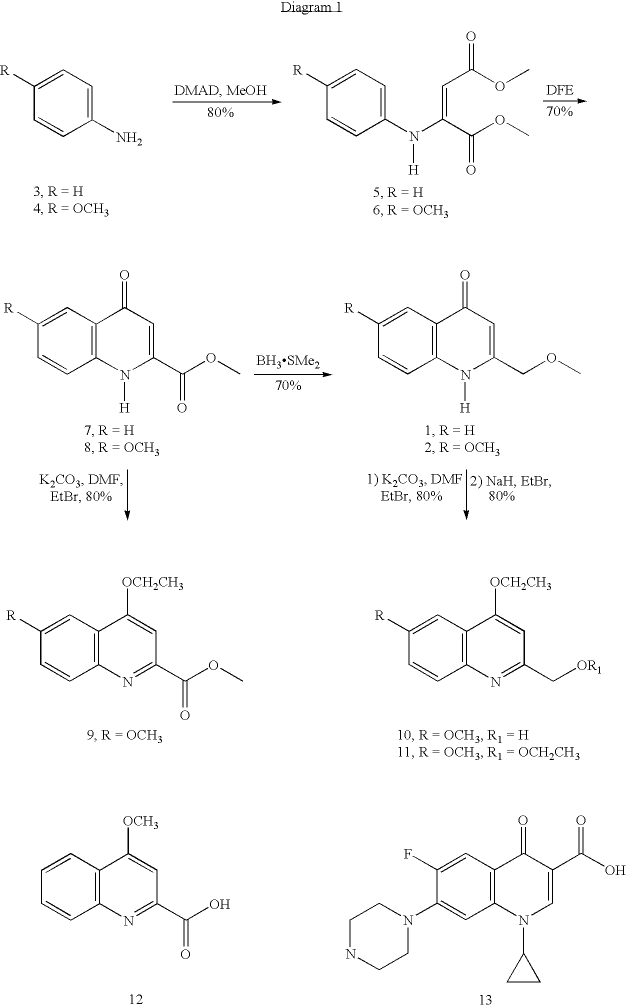

The details of each stage are explained below.

### Preparation of 2-(phenylamine)-dimethyl maleate or 2-anilin-2-dimethyl butenodioate (5) and 2-(4-methoxyphenylamine)-dimethyl maleate or 2-(4-methoxyanilin)-2-dimethyl butenodioate (6)

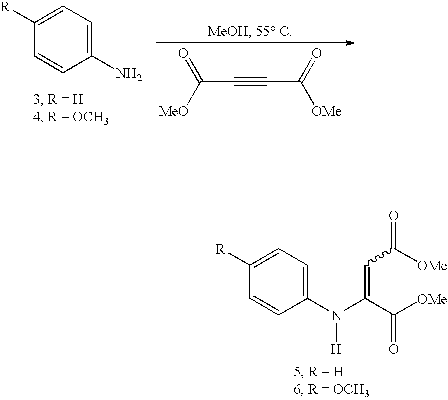

The enamines 5 and 6 are obtained based on the methodology described by Edmont, D.; Rocher, R.; Plisson, C. & Chenault, J. “Synthesis and evaluation of quinoline carboxyguanidines as antidiabetic agents” Bioorg. Med. Chem. Lett., 10: 1831, 2000, with a 50% yield.

The preparation process of the compounds 5 and 6 consists of the following experimental procedure:

A balloon flask containing anilin 3 (5.0 g, 0.054 mol) in dry MeOH (54 mL), has dimethyl acetylenodicarboxylate (DMAD) (7.7 g, 0.054 mol) added under N₂ at 55° C. The reaction is monitored by Fine Layer Chromatography (FLC) using n-hexane as an eluent. At the end of the reaction the MeOH is evaporated, after which CH₂Cl₂ (30 mL) is added for extraction and the organic phase is washed in a saturated solution of NH₄Cl (3×10 mL) followed by water (3×10 mL). The organic phase is dried and evaporated. Compound 5 is obtained with a yield of 50% (6.3 g).

RMN ¹H (200 MHz, CDCl₃) δ: 9.67 (sl, 1H); 7.32-7.25 (m, 2H); 7.13-7.05 (m, 1H); 6.92-6.88 (m, 2H); 5.39 (s, 1H); 3.74 (s; 3H); 3.70 (s, 3H).

RMN ¹³C (50 MHz, CDCl₃) δ: 169.75; 164.72; 147.91; 140.18; 129.03; 124.13; 120.60; 116.56; 93.46; 52.62; 51.07.

EM (relative intensity %) m/z: 235.25 (6.9); 144.15 (93); 77.10 (100).

IV (νₘₐₓ, KBr) cm⁻¹: 3457; 3380; 2953; 1739; 1668; 1282; 1031.

A balloon flask containing anisiline 4 (5.0 g, 0.041 mol) in dry MeOH (41 mL), has (DMAD) (5.8 g, 0.041 mol) added under N₂ at 55° C. The reaction is monitored by (FLC) using n-hexane as an eluent. At the end of the reaction the MeOH is evaporated, after which CH₂Cl₂ (30 mL) is added for extraction and the organic phase is washed in a saturated solution of NH₄Cl (3×10 mL) followed by water (3×10 mL). The organic phase is dried and evaporated. Compound 6 is obtained with a yield of 50% (5.5 g).

RMN ¹H (200 MHz, CDCl₃) δ: 9.57 (sl, 1H); 6.91-679 (m, 4H); 5.30 (s, 1H); 3.78 (s, 3H); 3.73 (s, 3H); 3.67 (s, 3H). RMN ¹³C (50 MHz, CDCl₃) δ: 182.44; 170.0; 164.76; 156.87; 148.99; 133.39; 122.96; 114.34; 91.66; 55.40; 52.61; 21.01.

IV (νₘₐₓ, KBr) cm⁻¹: 3284; 3210; 2952; 2836; 1742; 1637; 1033.

EM (relative intensity %) m/z: 265.50 (13); 146.15 (75); 77.15 (95).

### Preparation of the 4-oxo-1,4-dihydro-2-quinoline methyl carboxylate (7) and 6-methoxy-4-oxo-1,4-dihydro-2-quinoline methyl carboxylate (8)

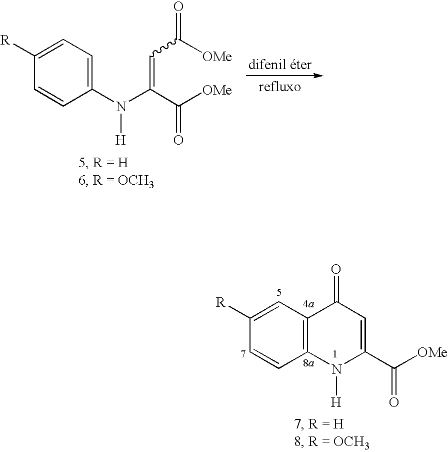

Intramolecular cyclisation is achieved through reaction at high temperature. The nucleophilic attack on the ester carbonyl is directed by the nitrogen which is an ortholpara director and thus closes the ring.

In laboratory, a balloon flask containing a reflux condenser has ether diphenyl (8 mL) and reflux added, after which enamine 5 (1 g, 4.25 mmol) is added and after a specific time this system is removed from the sand bath and immersed in ice with the precipitation of the substrates being observed. A pre-purification is performed using dry-flash separation with a gradient elution of n-hexane to methanol. Compound 7 is obtained with a yield of 70% (600 mg) following recrystallisation.

P.F.=215-225° C.

RMN ¹H (400 MHz, CDCl₃) δ: 12.03 (s, 1H); 8.13 (d, J=8.0 Hz, 1H); 7.95 (d, J=10 Hz, 1H); 7.66-7.63 (m, 1H); 7.35-7.32 (m, 1H); 6.73 (s; 1H); 3.99 (s; 3H).

RMN ¹³C (100 MHz, CDCl₃) δ: 176.39; 161.09; 138.44; 135.75; 130.55; 124.37; 123.04; 122.09; 117.89; 108.77; 51.53.

IV (νₘₐₓ, KBr) cm⁻¹: 3436; 2925; 2886; 1733; 1639; 1538; 1033.

EM (relative intensity %) m/z: 203 (30); 143 (100); 115.15 (73); 89.15 (66).

In a balloon flask containing a reflux condenser ether diphenyl (8 mL) was added, refluxed, after which enamine 6 (1.0 g, 3.8 mmol) was added and after a determined time the reflux was ended and the balloon was immersed in ice, observing the precipitation of the substrates. A pre-purification is performed using dry-flash separation with a gradient elution of hexane to methanol. Compound 8 is obtained with a yield of 60% (530 mg) following recrystallisation.

P.F.=250-260° C.

RMN ¹H (400 MHz, CDCl₃) δ: 12.14 (s, 1H); 7.91 (d, J=9.1 Hz, 1H); 7.46 (d, J=2.8 Hz, 1H); 7.66 (dd, J=9.0 e 2.8 Hz, 1H); 6.67 (s, 1H); 3.96 (s, 3H); 3.85 (s, 3H).

RMN ¹³C (100 MHz, CDCl₃) δ: 176.30; 162.28; 155.78; 136.18; 134.16; 126.73; 122.84; 120.91; 108.34; 103.19; 54.94; 52.90.

IV (νₘₐₓ, KBr) cm⁻¹: 3440; 2935; 2865; 1729; 1639; 1552; 1024.

EM (relative intensity %) m/z: 207.50 (54); 173.2 (91); 73.2 (100).

The compounds obtained were characterised using RMN ¹H and ¹³C and it was observed that one of the methoxyls of enamines 5 and 6 disappeared. The shift rate of the ester methoxyls to the closed system was at 3.99 and 3.96 ppm respectively for compounds 7 and 8.

The hydrogen in position 3 shifted at 6.73 and 6.67 ppm respectively for 5 and 6. It is possible to observe the hydrogens linked to the nitrogens (position 1) at 12.03 and 12.14 ppm respectively for 7 and 8.

The presence of methoxyl modifies the coupling systems in the aromatic system and in the case of 7 ortho couplings with J at 8.0 and 10 Hz are observed with the signal at 8.14 ppm (J=8.0 Hz) referring to H 9 while H 6 shows a shift at 7.97 ppm (J=10.0 Hz), H 8 shows a shift at 7.35 ppm while the H 7 signal is observed at 7.66 ppm.

The spectrum of RMN ¹³C for 7 shows a carbonyl signal (in carbon C4) α,β-insaturated at 176.39 ppm while C3 shows a shift at 108.76 ppm.

However, in the case of compound 8, a duplicate was observed at 7.9 ppm with J=9 Hz referring to H 8 and the dd at 7.39 ppm, with J of 9.0 and 2.8 Hz referring to H 7, since it has an ortho coupling with H 8 and a meta coupling with H 6. The spectrum of RMN ¹³C for 7 shows a carbonyl (C4) α,β-insaturated at 176.29 ppm while C3 shows a shift at 103.76 ppm.

### Preparation of 2-hydroxymethyl-4-quinolinone (1) and 2-hydroximethyl-6-methoxy-4-quinolinone (2)

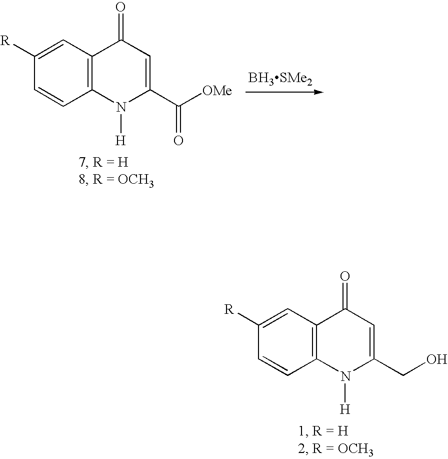

Selective reductions of functional carbonyl groups are important reactions in organic synthesis and several reducer agents have been developed. The borane-dimethyl sulphide (BH₃.5Me₂) complex is used in the reduction of esters with a strong preference for the group located in the a position of the hydroxyl groups (Saito, S., Ishikawa, T., Kuroda, A., Koga, K. & Moriwake, T. “A revised mechanism for chemoselective reduction of esters with borane-dimethyl sulfide complex and catalytic sodium tetrahydroborate directed by adjacent hydroxyl group”. Tetrahedron, 48: 4067, 1992).

It is acknowledged that the reduction of ketones in the presence of enones is possible using BH₃.5Me₂, while the ester group may be reduced with diisobutyl aluminium hydride (DIBAL-H) in the presence of enones (Larock, R. C. “Comprehensive organic transformations” A guide to functional group preparations p 537, 1989). There does not seem to be any prior mention in the literature for the type of system present in compounds 7 and 8.

Therefore, the research required for the present invention was initially directed at testing reduction using BH₃.5Me₂ due to the simplicity of the work-up for this reagent when compared to DIBAL-H since borane may be removed from the reagent medium through distillation with anhydrous MeOH without the addition of water and it is also possible to remove any other impurities through recrystallisation.

The reduction reaction of the ester group to the hydroxyl group using borane proves to be chemoselective and it is possible to obtain both compounds 1 and 2 with a yield of 70%.

Experimentally, a balloon flask containing compound 7 (500 mg, 2.5 mmol) in anhydrous THF (10 mL) under N₂ atmosphere at 0° C. has pure BH₃.5Me₂ complex (233 μL, 2.5 mmol) in a solution of THF (3 mL) added drop-by-drop. When the entire solution has been added, the reactor flask is left at room temperature. The reaction is monitored by FLC using AcOEt as eluent. Anhydrous MeOH (10 mL) is then added after 24 hours of reaction and the solution is distilled. Anhydrous MeOH (3×15 mL) is then added again. Compound 1 is obtained with a characterized yield of 70% (300 mg).

P.F.=230-240° C.

RMN ¹H (200 MHz, CDCl₃) δ: 8.04 (d, J=8 Hz, 1H); 7.69-7.56 (m, 2H); 7.31-7.23 (m, 1H); 6.02 (s, 1H); 4.48 (s, 2H).

RMN ¹³C (50 MHz, CDCl₃) δ: 153.49; 140.42; 131.56; 125.25; 124.95; 122.88; 118.59; 105.59; 60.47.

IV (νₘₐₓ, KBr) cm⁻¹: 3384; 2946; 2917; 1619; 1359; 1083.

A balloon flask containing compound 8 (500 mg, 2.16 mmol) in anhydrous THF (10 mL) under N₂ atmosphere at 0° C. has pure BH₃.5Me₂ complex (204 μL, 2.16 mmol) in a solution of THF (2 mL) added drop-by-drop. When the entire solution has been added, the reactor flask is left at room temperature. The reaction is monitored by FLC using AcOEt as eluent. Anhydrous MeOH (10 mL) is then added after 24 hours of reaction and the solution is distilled. Anhydrous MeOH (3×15 mL) is then added again, since the distillation process removes the remaining residues and impurities of BH₃SMe₂. Compound 2 is obtained with a characterized yield of 70% (300 mg).

P.F.=250-260° C.

RMN ¹H (400 MHz, CDCl₃) δ: 7.95-7.92 (m, 1H); 7.52-7.41 (m, 2H); 6.63 (s, 1H); 4.72 (s, 2H); 3.89 (s, 3H).

RMN ¹³C (100 MHz, CDCl₃) δ: 171.50; 156.79; 155.51; 134.45; 124.16; 123.15; 120.97; 103.11; 102.71; 60.00; 55.61.

IV (νₘₐₓ, KBr) cm⁻¹: 3448; 2921; 2852; 1637; 1504; 1035.

The compounds are characterised by RMN ¹H, whereby the forming of compound 1 is confirmed by the disappearance of the methoxyl group signal (3.99 ppm) and the appearance of the carbonylic methylene signal at 4.48 ppm, while the carbonylic methylene signal appears on the spectrum of RMN ¹³C at 60.47 ppm.

In the case of compound 2, the signal appearing at 4.72 ppm relates to carbinolic methylene and the disappearance pf methoxyl at 3.96 ppm.

### Preparation of 4-ethoxy-6-methoxy-2-quinoline methyl carboxylate (9) and 4-ethoxy-6-methoxy-2-quinolilmethanol (10)

The preparation of the quinolinic derivates occurs by a fast and clean reaction using DMF as solvent and K₂CO₃ as base and, depending on the intended product, using the alkylating agents EtBr or MeI (Edmont, D.; Rocher, R.; Plisson, C. & Chenault, J. “Synthesis and evaluation of quinoline carboxyguanidines as antidiabetic agents” Bioorg. Med. Chem. Lett., 10: 1831, 2000). The balance is shifted to form the O-alkylated product, with the selectivity depending on factors such as the alkyl halid structure, ring substitutes and the solvent (Comins, D. L. & Jianhua, G. “N- vs O-alkylation in the Mitsunobu reaction of 2-pyridone”. Tetrahedron Lett., 35: 2819, 1994.). All compounds are obtained using the same reaction conditions and the yields were moderate to good (60-80%).

A flask containing compound 8 (20 mg, 0.1 mmol) and K₂CO₃ (20.7 mg, 0.15 mmol) has added anhydrous DMF (50 μL) and MeI (9.5 μL, 0.15 mmol). The flask is agitated for a period of 12 hours at ambient temperature. The solution is then filtered using silica so as to remove the precipitate and the concentrated solvent following which the resulting material is purified using column chromatography using a gradient of n-hexane—methanol as eluent resulting in 17 mg of product 9 (80% yield).

P.F.: 105-107° C.

RMN ¹H (400 MHz, CDCl₃) δ: 8.11 (d, J=9.2 Hz, 1H); 7.55 (s, 1H); 7.46 (d, J=2.8 Hz, 1H); 7.48 (dd, J=9.2, 2.8 Hz, 2H); 4.36 (q, J=7 Hz, 2H); 4.06 (s, 3H); 3.96 (s, 3H); 1.60 (t, J=7 Hz, 3H).

RMN ¹³C (100 MHz, CDCl₃) δ: 166.46; 161.29; 158.91; 146.60; 144.42; 131.82; 123.42; 123.02; 101.01; 99.61; 64.53; 55.64; 53.08; 14.48.

IV (νₘₐₓ, KBr) cm⁻¹: 2933; 2856; 1730; 1639; 1483; 1236; 1024

Compound 10 is produced following a procedure analogous to that described above for compound 9.

The compound 4-ethoxy-2-ethoxymethyl-6-methoxyquinoline (11) is prepared using NaH in DMF and DME to ascertain the influence of the hydroxyl group on the biological activity (Osornio, Y. M.; Miranda, L. D.; Cruz-Almanza, R. & Muchowski, J. M. “Radical cyclizations to quinolone and isoquinolone systems under oxidative and reductive reductions” Tetrahedron. Lett., 45:2855, 2004.).

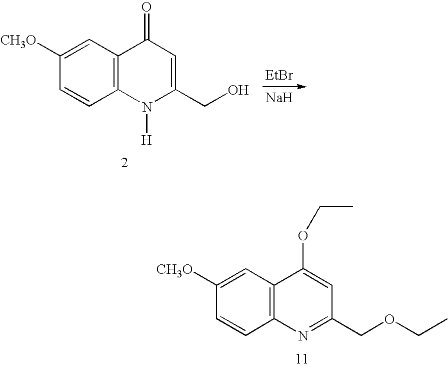

Compound 11 is characterised by RMN ¹H and its formation is confirmed by the appearance of two carbonillic methylene signs at 4.33 and 3.67 ppm apart from the methyls at 1.59 and 1.31 ppm, while the spectrum of RMN ¹³C shows the appearance of carbonillic methylenes at 74.39; 66.35; 64.09 ppm.

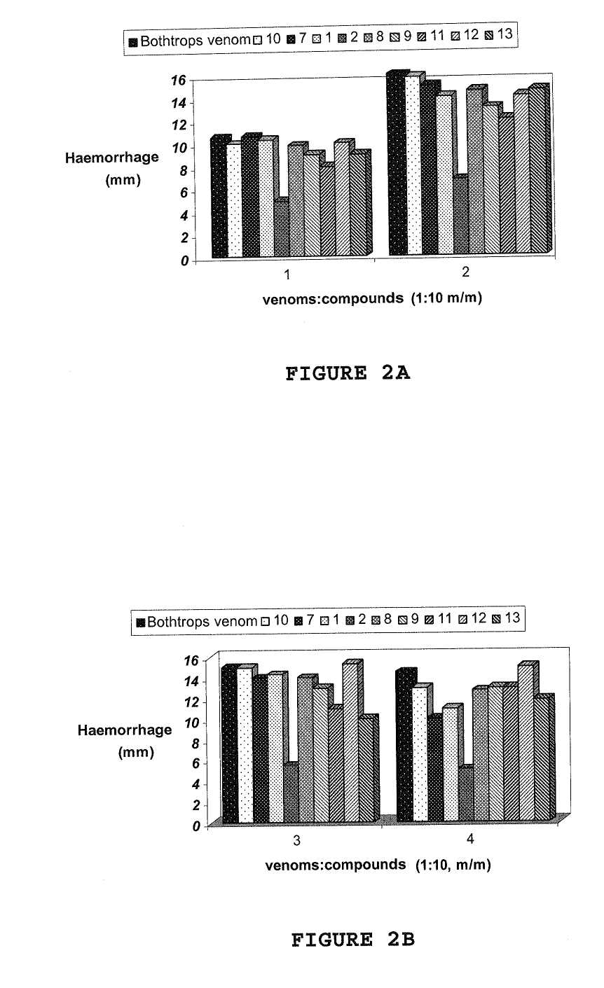

The compounds selected for the initial screening are shown in FIG. 2 in annex. This screening was for the venoms of the Bothrops jararacussu, B. moojeni, B. alternatus and B. jararacussu BjussuMP-I snakes to determine promising compounds.

This first assay established that 10, 1, 2, 11 and 13 presented partial inhibition properties over coagulant serine proteases with 2 being outstanding due to the added potential of inhibiting the haemorrhagic metalloprotease of the venoms and thus proving the most promising of the tested compounds illustrated in FIG. 2.

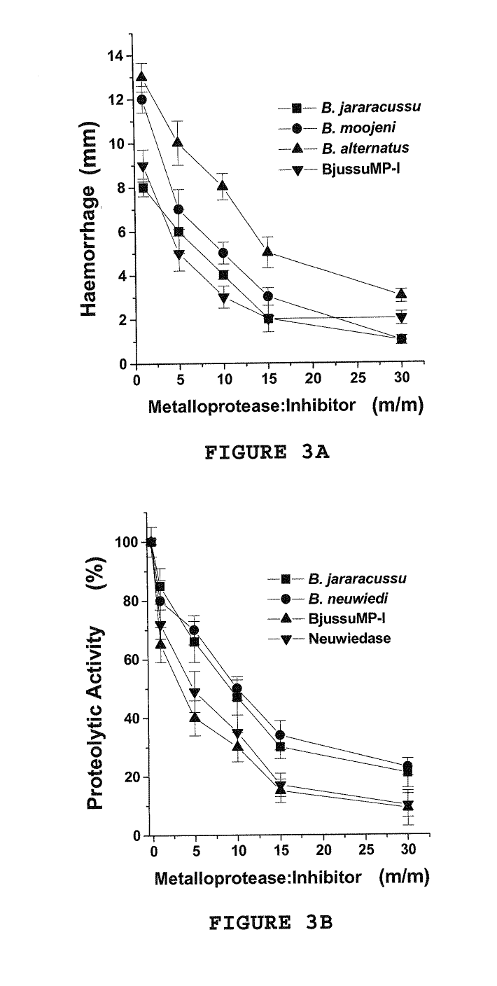

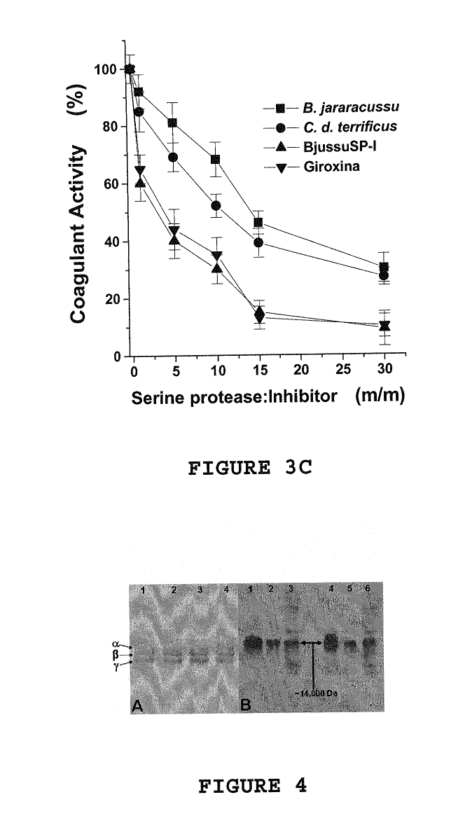

Therefore, further assays were performed to determine the efficiency of compound 2 for various other activities. FIG. 3A presents the effects of 2 on haemorrhages induced by the different venoms of the Bothrops genus snakes and an isolated metalloprotease.

The inhibition of haemorrhagic activity suggests interaction of the inhibitor with a metal and/or metalloprotease, thus neutralising effects. Likewise, compound 2 significantly inhibits proteolytic activity on casein and coagulants in human plasma induced by snake venoms and isolated enzymes, metalloprotease (FIG. 3B) or serine protease (FIG. 3C), respectively.

The proteolytic activity induced by Class I (neuwiedase isolated from B. neuwiedi) and III (BjussuMP-I isolated from B. jararacussu) metalloproteases was inhibited by compound 2 by approximately 67 and 70%, respectively, at a ratio of 1:10 protease:inhibitor (m/m).

The results for compound 2 show that it displays powerful coagulation action for the venoms of B. jararacussu and C. d. terrificus with this activity probably being due to the interaction of the active principle with the thrombin type enzymes BjussuSP-I and gyroxin respectively isolated from these same venoms (FIG. 3C).

The inhibition of fibrinogenolytic activity induced by serine protease and metalloprotease enzymes is shown in FIG. 4A. However, the mechanism of these compounds remains unknown but, nevertheless, SDS-PAGE techniques reveal no proteolytic degradation of the venom proteins as can be seen in FIG. 4B.

FIG. 4A shows the inhibition of fibrinogenolytic activity by the compound. SDS-PAGE shows the proteolytic activity on bovine fibrinogen caused by the venom of the B. jararacussu snake and isolated proteases. Lanes: 1—Fibrinogen+venom of B. jararacussu (4 μg)+2 (40 μg); 2—Fibrinogen+enzyme BjussuSP-I (2 μg)+2 (20 μg); 3—Fibrinogen+BjussuMP-I (2 μg)+2 (20 μg); 4—Fibrinogen control. The enzymes were incubated together with 2 for 30 minutes at 37° C. The results are expressed by the mean±S.D (n=3). (B): Interaction between the venom of the B. jararacussu snake and 2. Samples containing venom/toxin (20 μg) and the 88 (600 μg) were incubated for 30 minutes at 37° C. at a ratio of 1:30 (w/w). Lanes: 1—BthTX-I+2; 2—BthTX-II+2; 3—venom of B. jararacussu+2; 4—only BthTX-I; 5—only BthTX-II; 6—only the venom of B. jararacussu.

In the assays for myotoxicity, oedema, and activity for phospholipase in vivo, compound 2 partially reduced these effects when induced by the venoms of B. jararacussu and C. durissus terrificus and isolated PLA₂s (A

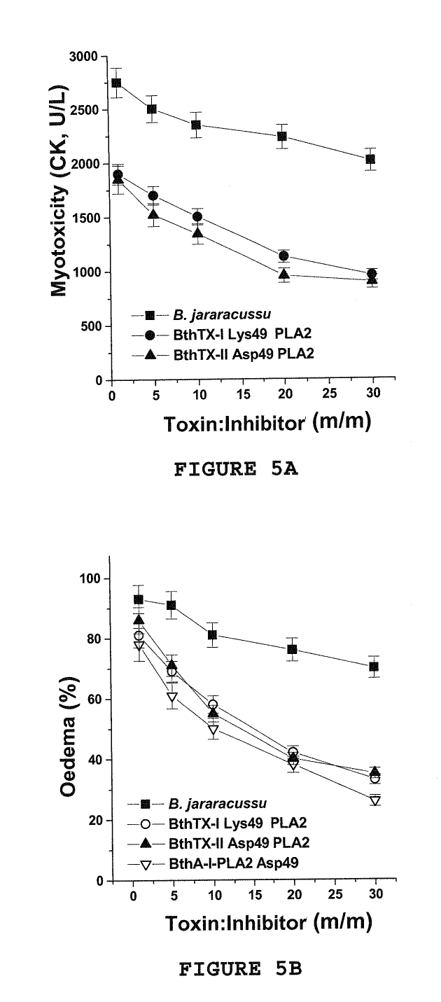

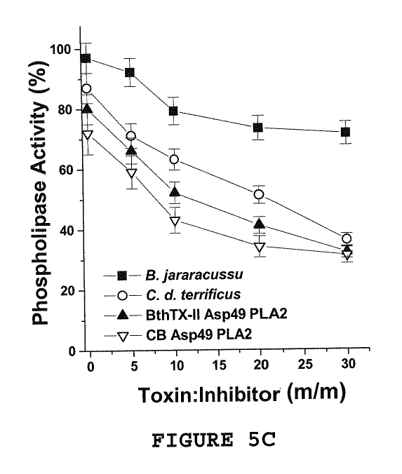

FIG. 5 shows the inhibitory activity for myotoxicity, oedema and phospholipase by compound 2. FIG. 5A shows the effect of 2 on the myotoxicity induced by the venom of B. jararacussu and isolated myotoxins (BthTX-I and II). FIG. 5B illustrates the effects of 2 on the oedems inducing activity caused by the venom of B. jararacussu and isolated PLA2s (basic Lys49 BthTX-I, basic Asp49 BthTX-II and acid Asp49 BthA-1-PLA2). FIG. 5C displays the phospholipase activity induced by the venom of B. jararacussu and C. d. terrificus and isolated PLA2s (BthTX-II and CB). The results are expressed by the mean±standard deviation (n=6).

The muscle damage inflicted by the venom of Bothrops is partially caused by a group of proteins having PLA₂ structures (Gutiérrez, J. M. & Lomonte, B. “Phospholipase A₂, myotoxins from Bothrops snake venoms”. Toxicon, 33:1405, 1995; Soares, A. M. & Giglio, J. R. Chemical modifications of phospholipases A₂ from snake venoms: Effects on catalytic and pharmacological properties. Review. Toxicon, 42: 855, 2003).

Compound 2 inhibited the myotoxic activity of both enzymes of Asp49 BthTX-II and Lys49 BthTX-I phospholipases A₂ of B. jararacussu. Compound 2 proved more efficient in neutralising PLA₂ activity induced by basic Asp49 PLA₂s (BthTX-II and CB) in A FIG. 5C than that induced by the pure venoms and the acid isoform Asp49 BthA-1-PLA₂.

These data suggest a more specific link with basic PLA₂s, intermediated by interactions of probable electrostatic cause and supports various authors who have pointed out the distinct or partial power of the overlap of catalytic sites and another pharmacologic one. (Soares, A. M. & Giglio, J. R. Chemical modifications of phospholipases A₂ from snake venoms: Effects on catalytic and pharmacological properties. Review. Toxicon, 42: 855, 2003.).

In conclusion, compound 2 inhibits haemorrhages, enhances coagulation, proteolytic activity, oedema and myotoxicity induced by the venom of the Bothrops and Crotalus snakes and isolated metalloprotease, serine protease and phospholipases A₂ enzymes demonstrating that the inhibitor is a good tool having potential antivenin activity.

The pharmacological efficiency of compound 2 is superior in inhibiting the proteases induced by PLA₂s and thus provided information for development of therapeutic agents for the treatment of haemostatic diseases. Furthermore, the inhibitor has potential use as a complementary antivenin and is an alternative for treating poisoning caused by snake bite.

Results have demonstrated that the compounds may be used as antirheumatics, analgesics, immunosuppressors, antivenins, antithrombotics, anti-allergics and expectorants as well as for the treatment of white blood cell disorders and haemostatic system disorders amongst other therapeutic applications; in illnesses related to white blood cell disorders, such as autoimmune and inflammatory diseases including rheumatism amongst others, as well as anti-coagulants, antivenin, analgesics, antithrombotics and other therapeutical applications.

In the assays for myotoxicity, oedema, and activity for phospholipase in vivo, compound 2 partially reduced these effects when induced by the venoms of B. jararacussu and C. durissus terrificus and isolated PLA₂s (A

FIG. 5 shows the inhibitory activity for myotoxicity, oedema and phospholipase by compound 2. FIG. 5A shows the effect of 2 on the myotoxicity induced by the venom of B. jararacussu and isolated myotoxins (BthTX-I and II). FIG. 5B illustrates the effects of 2 on the oedems inducing activity caused by the venom of B. jararacussu and isolated PLA2s (basic Lys49 BthTX-I, basic Asp49 BthTX-II and acid Asp49 BthA-1-PLA2). FIG. 5C displays the phospholipase activity induced by the venom of B. jararacussu and C. d. terrificus and isolated PLA2s (BthTX-II and CB). The results are expressed by the mean±standard deviation (n 6).

The muscle damage inflicted by the venom of Bothrops is partially caused by a group of proteins having PLA₂ structures.

Compound 2 inhibits the myotoxic activity of both enzymes Asp49 BthTX-II and Lys49 BthTX-I phospholipases A₂ of B. jararacussu. Compound 2 proved more efficient in neutralising PLA₂ activity induced by basic Asp49 PLA₂s (BthTX-II and CB) in A FIG. 5C than that induced by the pure venoms and the acid isoform Asp49 BthA-1-PLA₂.

FIG. 5 shows the inhibitory activity for myotoxicity, oedema and phospholipase by compound 2. FIG. 5A shows the effect of 2 on the myotoxicity induced by the venom of B. jararacussu and isolated myotoxins (BthTX-I and II). FIG. 5B illustrates the effects of 2 on the oedems inducing activity caused by the venom of B. jararacussu and isolated PLA2s (basic Lys49 BthTX-I, basic Asp49 BthTX-II and acid Asp49 BthA-1-PLA₂). FIG. 5C displays the phospholipase activity induced by the venom of B. jararacussu and C. d. terrificus and isolated PLA₂s (BthTX-II and CB). The results are expressed by the mean±standard deviation (n=6).

This data suggests a more specific link with basic PLA₂s, intermediated by interactions of probable electrostatic cause and supports various authors who have pointed out the distinct or partial power of the overlap of the catalytic sites and another pharmacologic one, as related in the article by Soares, A. M. & Giglio, J. R. Chemical modifications of phospholipases A₂ from snake venoms: Effects on catalytic and pharmacological properties. Review. Toxicon, 42: 855, 2003.

Therefore, compound 2 inhibits haemorrhages, enhances coagulation, proteolytic activity, oedema and myotoxicity induced by the venom of the Bothrops and Crotalus snakes and isolated metalloprotease, serine protease and phospholipases A₂ enzymes demonstrating that the inhibitor is a good tool having potential antivenin activity.

The compounds were tested against snake venom in accordance with the procedures described below:

### Proteolytic Activity in Casein

Snake venom or isolated metalloprotease (40 μg) is incubated with casein 1% (w/v) (1.0 mL) in a buffer solution of 0.1 M Tris-HCl (pH 8.0) for 30 minutes at 37° C. The reaction is ended through the addition of a trichloroacetic acid solution at 5% (v/v) (1.0 mL) and the mixture is left standing for 30 minutes at ambient temperature and then centrifuged (2000 rpm) for 5 minutes at 25° C. The proteolytic activity is estimated by the measurement of the absorbance of the supernatant at 280 nm.

### Coagulant Activity

Aliquots of plasma (0.2 mL) were incubated with venom or venom/inhibitor (50 μL) in varying proportions (m/m) during a period of 30 minutes at 37° C. and the coagulation time was verified. The control tubes included plasma incubated with phosphate buffered saline solution (PBS)+calcium or dimethyl sulphoxide or only the compound.

### Oedema Inducing Activity

The oedemas are induced by the direct injection of venom (20 μg) and purified proteins (20 μg) into the right leg of male Swiss mice (18-22 g). Inhibition activity is verified incubating the venom or isolated protein with the inhibitor in varying concentrations (m/m). The control groups are injected with 50 μL of phosphate buffered saline solution (PBS, pH=7.2) alone, dimethyl sulphoxide (DMSO) or the compounds. Oedema progression is assessed through measurement of the decrease in blood pressure using a pachymeter (Mitutoyo, Japan) at intervals of 30 and 60 minute after injection.

### Myotoxic Activity

Male Swiss mice (18-22 g) are injected with solutions containing 25 μg/50 μL doses of venom or toxin intra-muscularly at the right leg. The mixtures of venom or toxin/inhibitor (m/m) are then verified. The controls receive phosphate buffered saline solution or just inhibitor. The mice are bled at the tail 3 hours after injection and the blood is collected in capillary tubes containing heparin. The creatin kinase (CK) activity of the plasma is determined using a Bioclin Kit (Bioclin, Brasil). This activity is expressed in units/L, a corresponding unit for the production of one micromol of nicotinamide adenine dinucleotide (NADH)/min at 30° C.

### Phospholipase A₂ Activity

Indirect haemolytic activity is tested using agarose-erythrocyte-egg yolk gel as a substrate. The compounds are tested following incubation with the pure venoms or PLA₂ in varying ratios (m/m).

The data was analysed according P the value of <0.05, which was considered as indicative of significance.

1. 4-quinolinones, comprising the formula (I) 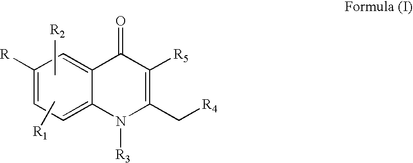
Whereby:
R═H or OCH₃
R₁ and R₂ are selected independently of each other, with H, OH, an alkyl group of C₁-C₄, an alkoxy group of C₁-C₅, a —OCO—R₇ group, and a group derived from a saccharide, optionally R₁ and R₂ together forming a methylenedioxy group, a phenyl group or a phenyl group substituted in 1 and 3 with groups selected from H, an alkoxy group in C₁-C₄, a —OCOR₇ group, a —O—SO₂—R₇ group, halogen, an alkyl or CF₃ group, and —NR₇R₈ group, in which R₇ and R₈ are selected independently of each other, from hydrogen, alkyl group in C₁-C₅, alkenyl group, alkyl phenyl group (C₁-C₄), dimethylamine, rings with 4-6 member heterocycles, optionally with one or more heteroatoms selected from oxygen, nitrogen and sulphur or a methylpiperazinyl group,
R₃ is selected from H, alkyl group in C₁-C₄, alkenyl group, a —CO—R₈ group and a -A-R₉ group, —CO₂R₉′ group in which R₉′ is a benzyl group, branched or linear alkyl group, p-methoxy benzyl group, —NH₂, —CHCH₂CH₂; R₈ is an alkyl group in C₁-C₄; A is an alkylene group in C₁-C₄; R₉ is selected from heterocycle groups with 5 or 6 members containing 1 to 4 heteroatoms of oxygen, sulphur and nitrogen, CN, hydroxyl, —COOR₁₀ and CONR₁₁R₁₂ groups, a —NR₁₃R₁₄ group, a —COR₁₅ group and a OSO₂R₁₆ group;
R₁₀, R₁₁, R₁₂, R₁₄ and R₁₅ are independently selected from hydrogen, alkyl groups in C₁-C₄, halogen and alkyl phenyl group (C₁-C₄), R₁₆ is selected from the phenyl group and the alkyl phenyl group (C₁-C₄);
R₄═OH, halogens, alkoxy group in C₁-C₆, alkoxy benzyl group, —CO—R₁₇ in which R₁₇ is alkyl C₁-C₆ or p-methoxy benzyl, —O—SO₂—R₇′ in which R₇′ is an alkyl group or CF₃ group, group derived from a saccharide; R₅ is H, halogen, phenyl group or phenyl substituted 1 or 3 times with groups selected from H, alkoxy groups C₁-C₄, a —OCOR₇ group, a —O—SO₂—R₇′ group in which R₇′ is an alkyl group or CF₃ group, benzylamine group, and group derived from saccharide, alkyl group, —COOH, or salts, hydrates and pharmacologically acceptable pro-pharmacons.

2. 4-quinolinones in accordance with claim 1, wherein R═H and R₄═OH (1) or, alternatively, R═OCH₃ and R₄═OH (2).

3. 4-quinolinones in accordance with claim 1, wherein said 4-quinolinones are used to inhibit proteases, lipases, phospholipases and enzymes.

4. 4-quinolinones in accordance with claim 1, wherein said 4-quinolinones are used in the treatment of inflammatory diseases, autoimmune diseases, as antirheumatic, analgesic, antivenin, antithrombotic, anti-allergic, expectorant, disorders of white blood cells, disorders of the haemostatic system, amongst other pharmaceutical applications.

5. Quinolines, comprising the formula (II): 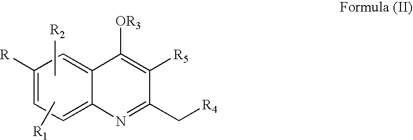
Whereby:
R═H or OCH₃
R₁ and R₂ are selected independently of each other, with H, OH, an alkyl group of C₁-C₄, an alkoxy group of C₁-C₅, a —OCO—R₇ group, and a group derived from a saccharide, optionally R₁ and R₂ together forming a methylenedioxy group, a phenyl group or a phenyl group substituted in 1 and 3 with groups selected from H, an alkoxy group in C₁-C₄, a —OCOR₇ group, a —O—SO₂—R₇ group, halogen, an alkyl or CF₃ group, and —NR₇R₈ group, in which R₇ and R₅ are selected independently of each other, from hydrogen, alkyl group in C₁-C₅) alkenyl group, alkyl phenyl group (C₁-C₄), dimethylamine, rings with 4-6 member heterocycles, optionally with one or more heteroatoms selected from oxygen, nitrogen and sulphur or a methylpiperazinyl group,
R₃ is selected from H, alkyl group in C₁-C₄, alkenyl group, a —CO—R₈ group and a -A-R₉ group, —CO₂R₉′ group in which R₉′ is a benzyl group, branched or linear alkyl group, p-methoxy benzyl group, —NH₂, —CHCH₂CH₂; R₈ is an alkyl group in C₁-C₄; A is an alkylene group in C₁-C₄; R₉ is selected from heterocycle groups with 5 or 6 members containing 1 to 4 heteroatoms of oxygen, sulphur, nitrogen, CN, hydroxyl, —COOR₁₀ and CONR₁₁R₁₂ groups, a —NR₁₃R₁₄ group, a —COR₁₅ group and a OSO₂R₁₆ group;
R₁₀, R₁₁, R₁₂, R₁₄ and R₁₅ are independently selected from hydrogen, alkyl groups in C₁-C₄, halogen and alkyl phenyl group (C₁-C₄), R₁₆ is selected from the phenyl group and the alkyl phenyl group (C₁-C₄),
R₄═OH, halogens, alkoxy group in C₁-C₆, alkoxy benzyl group, —CO—R₁₇ in which R₁₇ is alkyl C₁-C₆ or p-methoxy benzyl, —O—SO₂—R₇′ in which R₇′ is an alkyl group or CF₃ group, group derived from saccharide; R₅ is H, halogen, phenyl group or phenyl substituted 1 or 3 times with groups selected from H, alkoxy groups C₁-C₄, a —OCOR₇ group, a —O—SO₂—R₇′ group in which R₇′ is an alkyl group or CF₃ group, benzylamine group, and group derived from saccharide, alkyl group, —COOH, or salts, hydrates and pharmacologically acceptable pro-pharmacons.

6. Quinolines in accordance with claim 5, wherein said quinolines are obtained from the reaction of the 4-quinolinone compounds of formula (I) with K₂CO₃ and alkylation agents.

7. Quinolines in accordance with claim 5, wherein R═OCH₃, R₃═CH₂CH₃, R₄═OHeR═OCH₃, R₃═CH₂CH₃, R₄═OCH₂CH₃.

8. Quinolines in accordance with claim 5, wherein said quinolines are used to inhibit proteases, lipases, phospholipases and enzymes.

9. Quinolines in accordance with claim 5, wherein said quinolines are used in the treatment of inflammatory diseases, autoimmune diseases, as antirheumatic, analgesic, antivenin, antithrombotic, anti-allergic, expectorant, disorders of white blood cells, disorders of the haemostatic system, amongst other pharmaceutical applications.

10. Process for the preparation of the 4-quinolinones in accordance with claim 1, from anilin or derivates in the presence of DMAD, the product is refluxed with diphenyl ether to obtain compounds 7 and 8, wherein the process that consists in making compounds 7 and 8 react with a borane-dimethyl sulphide complex (BH₃.5Me₂), obtaining the compounds 2-hydroxymethyl-4-quinolinone 1 and 2-hydroxymethyl-6-methoxy-4-quinolinone 2.

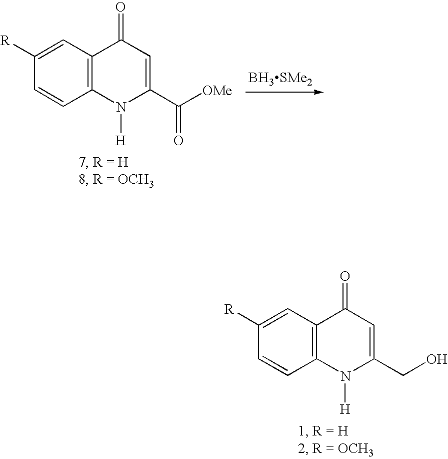

11. Process in accordance with claim 10, wherein said process uses groups comprising acids, aldehydes, amides, small chain esters (C₁-C₅), as initial material in the reduction with borane, while the group of solvents used in the reaction comprise ethers, THF and dioxane, hydrocarbonate solvents including toluene, benzene and halogenised hydrocarbonates, dichloromethane, chloroform, 1,2-dichloroethane.

12. Process in accordance with claim 10, wherein said process uses reductor agents, such as BH₃SMe₂, BH₃, DIBAL-H.

13. Pharmaceutical formulations, comprising an effective amount of the compound of formula (I) in accordance with claim 1 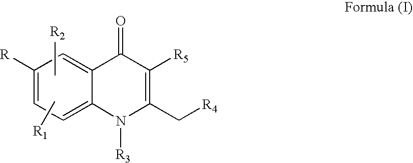
Whereby:
R═H or OCH₃
R₁ and R₂ are selected independently of each other, with H, OH, an alkyl group of C₁-C₄, an alkoxy group of C₁-C₅, a —OCO—R₇ group, and a group derived from a saccharide, optionally R₁ and R₂ together forming a methylenedioxy group, a phenyl group or a phenyl group substituted in 1 and 3 with groups selected from H, an alkoxy group in C₁-C₄, a —OCOR₇ group, a —O—SO₂—R₇ group, halogen, an alkyl or CF₃ group, and —NR₇R₈ group, in which R₇ and R₈ are selected independently of each other, from hydrogen, alkyl group in C₁-C₅, alkenyl group, alkyl phenyl group (C₁-C₄), dimethylamine, rings with 4-6 member heterocycles, optionally with one or more heteroatoms selected from oxygen, nitrogen and sulphur or a methylpiperazinyl group,
R₃ is selected from H, alkyl group in C₁-C₄, alkenyl group, a —CO—R₈ group and a -A-R₉ group, —CO₂R₉′ group in which R₉′ is a benzyl group, branched or linear alkyl group, p-methoxy benzyl group, —NH₂, —CHCH₂CH₂; R₈ is an alkyl group in C₁-C₄; A is an alkylene group in C₁-C₄, R₉ is selected from heterocycle groups with 5 or 6 members containing 1 to 4 heteroatoms of oxygen, sulphur, nitrogen,
CN, hydroxyl, —COOR₁₀ and CONR₁₁R₁₂ groups, a —NR₁₃R₁₄ group, a —COR₁₅ group and a OSO₂R₁₆ group;
R₁₀, R₁₁, R₁₂, R₁₄ and R₁₅ are independently selected from hydrogen, alkyl groups in C₁-C₄, halogen and alkyl phenyl group (C₁-C₄), R₁₆ is selected from the phenyl group and the alkyl phenyl group (C₁-C₄);
R₄═OH, halogens, alkoxy group in C₁-C₆, alkoxy benzyl group, —CO—R₁₇ in which R₁₇ is alkyl C₁-C₆ or p-methoxy benzyl, —O—SO₂—R₇′ in which R₇′ is an alkyl group or CF₃ group, group derived from saccharide; R₅ is H, halogen, phenyl group or phenyl substituted 1 or 3 times with groups selected from H, alkoxy groups C₁-C₄, a —OCOR₇ group, a —O—SO₂—R₇′ group in which R₇′ is an alkyl group or CF₃ group, benzylamine group, and group derived from saccharide, alkyl group, —COOH, or salts, hydrates and pharmacologically acceptable pro-pharmacons.

14. Pharmaceutical formulations in accordance with claim 13, comprising an effective amount of a compound whereby R═H and R₄═OH or alternatively whereby R═OCH₃ and R₄═OH.

15. Pharmaceutical formulations in accordance with claim 13, wherein said pharmaceutical formulations are used to inhibit proteases, lipases, phospholipases and enzymes.

16. Pharmaceutical formulations in accordance with claim 13, wherein said pharmaceutical formulations are used in the treatment of inflammatory diseases, autoimmune diseases, as antirheumatic, analgesic, antivenin, antithrombotic, anti-allergic, expectorant, disorders of white blood cells, disorders of the haemostatic system, amongst other pharmaceutical applications.

17. Pharmaceutical formulations, comprising an effective amount of the compound of formula (II) in accordance with claim 5 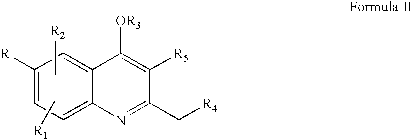
Whereby:
R═H or OCH₃
R₁ and R₂ are selected independently of each other, with H, OH, an alkyl group of C₁-C₄, an alkoxy group of C₁-C₅, a —OCO—R₇ group, and a group derived from a saccharide, optionally R₁ and R₂ together forming a methylenedioxy group, a phenyl group or a phenyl group substituted in 1 and 3 with groups selected from H, an alkoxy group in C₁-C₄, a —OCOR₇ group, a —O—SO₂—R₇ group, halogen, an alkyl or CF₃ group, and —NR₇R₈ group, in which R₇ and R₈ are selected independently of each other, from hydrogen, alkyl group in C₁-C₅, alkenyl group, alkyl phenyl group (C₁-C₄), dimethylamine, rings with 4-6 member heterocycles, optionally with one or more heteroatoms selected from oxygen, nitrogen and sulphur or a methylpiperazinyl group;
R₃ is selected from H, alkyl group in C₁-C₄, alkenyl group, a —CO—R₈ group and a -A-R₉ group, —CO₂R₉′ group in which R₉′ is a benzyl group, branched or linear alkyl group, p-methoxy benzyl group, —NH₂, —CHCH₂CH₂; R₈ is an alkyl group in C₁-C₄; A is an alkylene group in C₁-C₄, R₉ is selected from heterocycle groups with 5 or 6 members containing 1 to 4 heteroatoms of oxygen, sulphur, nitrogen, CN, hydroxyl, —COOR₁₀ and CONR₁₁R₁₂ groups, a —NR₁₃R₁₄ group, a —COR₁₅ group and a OSO₂R₁₆ group;
R₁₀, R₁₁, R₁₂, R₁₄ and R₁₅ are independently selected from hydrogen, alkyl groups in C₁-C₄, halogen and alkyl phenyl group (C₁-C₄), R₁₆ is selected from the phenyl group and the alkyl phenyl group (C₁-C₄);
R₄═OH, halogens, alkoxy group in C₁-C₆, alkoxy benzyl group, —CO—R₁₇ in which R₁₇ is alkyl C₁-C₆ or p-methoxy benzyl, —O—SO₂—R₇′ in which R₇′ is an alkyl group or CF₃ group, group derived from saccharide; R₅ is H, halogen, phenyl group or phenyl substituted 1 or 3 times with groups selected from H, alkoxy groups C₁-C₄, a —OCOR₇ group, a —O—SO₂—R₇′ group in which R₇′ is an alkyl group or CF₃ group, benzylamine group, and group derived from saccharide, alkyl group, —COOH, or salts, hydrates and pharmacologically acceptable pro-pharmacons.

18. Pharmaceutical formulations in accordance with claim 17, comprising an effective amount of a compound whereby R═OCH₃, R₃═OCH₂CH₃, R₄═OCH₂CH₃ or alternatively whereby R═OCH₃, R₃═OCH₂CH₃, R₄═OH.

19. Pharmaceutical formulations in accordance with claim 17, comprising an effective amount of a compound whereby R═OCH₃, R₃═OCH₂CH₃, R₄═OCH₂CH₃ or alternatively, whereby R═OCH₃, R₃═OCH₂CH₃, R₄═OH.

20. Pharmaceutical formulations in accordance with claim 17 wherein said pharmaceutical formulations are used to inhibit proteases, lipases, phospholipases and enzymes.

21. Pharmaceutical formulations in accordance with claim 17, wherein said pharmaceutical formulations are used in the treatment of inflammatory diseases, autoimmune diseases, as antirheumatic, analgesic, antivenin, antithrombotic, anti-allergic, expectorant, disorders of white blood cells, disorders of the haemostatic system, amongst other pharmaceutical applications.

22. Pharmaceutical formulations in accordance with claim 1 comprising: a) a compound of general formula (I) as active principle used in a quantity of not less than 0.001% by weight of the final composition, and b) not less than one pharmaceutically appropriate excipient.

23. Pharmaceutical formulations in accordance with claim 5 comprising: a) a compound of general formula (II) as active principle used in a quantity of not less than 0.001% by weight of the final composition, and b) not less than one pharmaceutically appropriate excipient.

24. Pharmaceutical formulations in accordance with claim 13, wherein said pharmaceutical formulations are administered by oral, rectal, topical or parenteral route.

25. Pharmaceutical formulations in accordance with claim 13, wherein said pharmaceutical formulations are prepared in the form of pills, coated pills, capsules, inhalable powder, effervescent tablets, sublingual pills, syrups and oral solutions, injectable solutions, ointments, creams, gels and other pharmaceutical preparations known in pharmaceutical techniques.

26. Use of 4-quinolinones and Quinolines in accordance with claim 1 comprising a composition, formulation or medicine to treat animal or human illnesses.
